(19) United States
(12) Patent Application Publication
(10) Pub. No.: US 2023/0157775 A1
(43) Pub. Date: May 25, 2023

(54) AUTONOMOUS ENERGY EXCHANGE SYSTEMS USING DOUBLE DYNAMIC MODEL

(71) Applicant: AutonomEES, Be'er Ya'akov (IL)

(72) Inventor: David Zeltzer, Tel Aviv (IL)

(21) Appl. No.: 18/057,606

(22) Filed: Nov. 21, 2022

Related U.S. Application Data
(60) Provisional application No. 63/282,086, filed on Nov. 22, 2021.

(51) Int. Cl. A61B 34/32; A61B 34/20

(52) U.S. Cl. CPC A61B 34/32; A61B 34/20; A61B 2034/2074

(57) ABSTRACT

Disclosed are actuators and power-train sub-systems of Autonomous Motion Systems (AMS), having a Double Dynamic Model (DDM) with combined functionality of control (including continuous controllability), operation (e.g., automated/robotic/autonomous operation in normal mode or safety mode) and learning using Energy Exchange System (EES) platform. DDM solution provides ability for an AMS to operate in real-world scenarios involving dynamic geometry and changing physical environment. For example, the first component can be an actuator and power train (A&P) system of an autonomous vehicle. The second component can be an autonomous simulation and test (AST) fixture on which a wheel of the autonomous vehicle is mounted, wherein the vehicle AST simulates a road or off-road condition for the autonomous vehicle. In another example, the first component can be a surgical A&P system of a robotic surgical device, and the second component can be a surgical AST that simulates an environment of living tissue.

### RELATED APPLICATIONS

This application is related to and claims the benefit of U.S. Provisional Patent Application No. 63/282,086, filed Nov. 22, 2021, titled “Autonomous Energy Exchange Systems Using Double Dynamic Model,” the entirety of which is incorporated herein by reference.

### TECHNICAL FIELD

Embodiments of the disclosure relate generally to a model used for autonomous energy exchange systems.

### BACKGROUND

One of the objectives in the field of Autonomous Motion Systems (also called Automatic/Robotic Motion Systems) is the ability of engage in safe and self-controlled behavior in different real-world situations and environments. Example of real-word environment include on-road and/or off-road situation for autonomous vehicles, open sea winds/wave/currents for marine vessels, and live tissue (like human organs) for robotic surgical devices. Accurate modeling of that behavior is critical for the success of such systems. This disclosure teaches a double dynamic model (DDM) based on the proprietary Energy Exchange System (EES) platform. Details of an EES can be found in U.S. Pat. No. 9,897,985, titled “Energy Exchange Systems Having Actuators with Multi-Parametric Control,” which is incorporated by reference herein in its entirety.

### SUMMARY

The following is a simplified summary of the disclosure in order to provide a basic understanding of some aspects of the disclosure. This summary is not an extensive overview of the disclosure. It is intended to neither identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

Generally, this disclosure describes control of an autonomous motion system (AMS) that operates in an “a priori unknown” environment that requires continuous observability. Control in dynamically changing environment requires continuous controllability and solution to convolution problems, which the Energy Exchange System (EES) is capable of. The EES is at the interface of two multi-parametric components with one or more active points. The active points require sensor-less and fast control. The combination of sensorless actuators (SA), EES and double dynamic model (DDM) described here is versatile enough to solve complex motion control problem for a variety of AMS.

Specifically, an aspect of the disclosure describes a system for autonomous motion control, comprising: a first component and a second component that interact with each other at one or more active points (also referred to as “work points”) at an interface of the first component and the second component, wherein each of the first component and the second component is represented by a respective dynamic multi-parametric model, wherein the first component is part of an autonomous motion system (AMS) and the second component simulates an environment in which the AMS operates in; an energy exchange platform at the interface that receives control data, operation data and learning data from each of the first component and the second component, and generates, by a processor at the energy exchange platform, a control vector associated with mechanical energy exchange at the one or more active points due to the interaction of the first component and the second component, wherein the processor further recalculates parameters of the respective dynamic multi-parametric models of the first component and the second component using the control vector as a baseline, thereby autonomously predicting and regulating dynamic behavior of the AMS.

In an embodiment, the first component can comprise a vehicle actuator and power train (vehicle A&P) system of an autonomous vehicle. The second component can comprise a vehicle autonomous simulation and test (vehicle AST) fixture on which a wheel of the autonomous vehicle is mounted, wherein the vehicle AST simulates a road or off-road condition for the autonomous vehicle.

In another embodiment, the first component can comprise a surgical actuator and power train (surgical A&P) system of a robotic surgical device, and the second component can comprise a surgical autonomous simulation and test (surgical AST) fixture that simulates an environment of living tissue in which the robotic surgical device is to perform.

### BRIEF DESCRIPTION OF THE DRAWINGS

The present disclosure will be understood more fully from the detailed description given below and from the accompanying drawings of various embodiments of the disclosure.

FIG. 1 illustrates an autonomous motion system including a navigation sub-system and an actuation sub-system, according to an embodiment of the present disclosure.

FIG. 2 illustrates an example of four wheels of a vehicle mounted on respective components of a test apparatus, according to an embodiment of the present disclosure.

FIG. 3 illustrates building blocks of a Double Dynamic Model (DDM), according to an embodiment of the present disclosure.

FIG. 3A illustrates a multi-functional DDM layer, according to an embodiment of the present disclosure.

FIG. 4 illustrates a generic functional configuration of an Energy Exchange System (EES) with DDM, according to an embodiment of the present disclosure.

FIG. 5 illustrates the basic parameters of a DDM, according to an embodiment of the present disclosure.

FIG. 5A illustrates a Central Integrated Unit (CIU) for a single active point (also referred to as “work point”), according to an embodiment of the present disclosure.

FIG. 6 illustrates a basic model for learning, according to an embodiment of the present disclosure.

FIG. 7 illustrates a triangular model for learning, according to an embodiment of the present disclosure.

FIG. 8 illustrates a block diagram of a Vehicle Actuators & Power-train (Vehicle A&P) system coupled to a CIU for each work point, according to an embodiment of the present disclosure.

FIG. 9 illustrates a mechanical structure of a Vehicle Autonomous Simulator/Tester (Vehicle AST) to generate work point data, according to an embodiment of the present disclosure.

FIG. 10 illustrates a multi-section Surgical Actuators & Power-train (Surgical A&P), according to an embodiment of the present disclosure.

FIG. 11 illustrates a Surgical Autonomous Simulator/Tester (Surgical AST) coupled to a Surgical Autonomous Operation System (AOS), according to an embodiment of the present disclosure.

FIG. 12 illustrates Simulation and Test (ST) commands calculation, according to an embodiment of the present disclosure.

FIG. 13 illustrates a flowchart describing a method for safety testing, according to an embodiment of the present disclosure.

### DETAILED DESCRIPTION

Embodiments of the present disclosure are directed to actuators and power-train sub-systems of autonomous/automatic motion systems, having Double Dynamic Model (DDM) with combined functionality of control, operation and learning, using Energy Exchange System (EES) platform. DDM solution provides ability for Autonomous Motion System to operate in real-world scenarios involving dynamic geometry and physical structures. DDM based Autonomous Motion Systems (AMS) contain two coupled components interacting with each other: 1) Autonomous Operation System (AOS), and 2) Autonomous Simulator/Tester (AST). Example of AOS include, but are not limited to, an autonomous vehicle, a marine vessel, a robotic surgical device etc. Specifically, the actuator and power train (A&P) of an autonomous motion system (AMS) can be the AOS, i.e. the first component of the two coupled components. The second component AST is coupled to A&P and simulates the physical environment that the first component operates in.

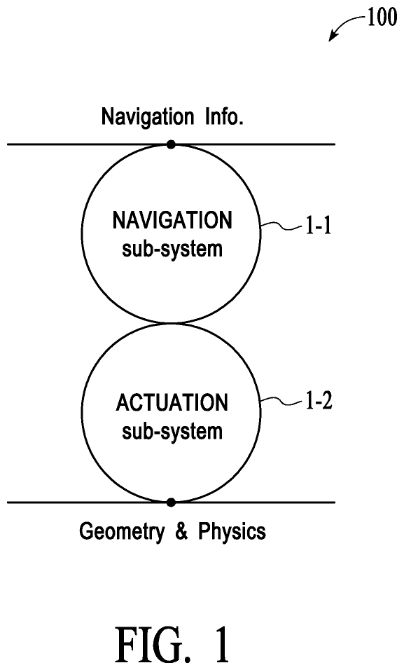

FIG. 1 shows the top-level set up of an Autonomous Operation System 100 for the specific case of an autonomous vehicle. AOS 100 comprises navigation sub-system (1-1) and actuation sub-system (1-2). Thus sub-systems interfaces are: navigation environment, actuation environment and navigation-to-actuation command and communication. Description below refers to actuation sub-system (1-2) only as the first component of the AMS. The actuation sub-system (1-2) contacts with physical areas (e.g., road or off-road) and/or with simulators of that simulates road/off-road conditions.

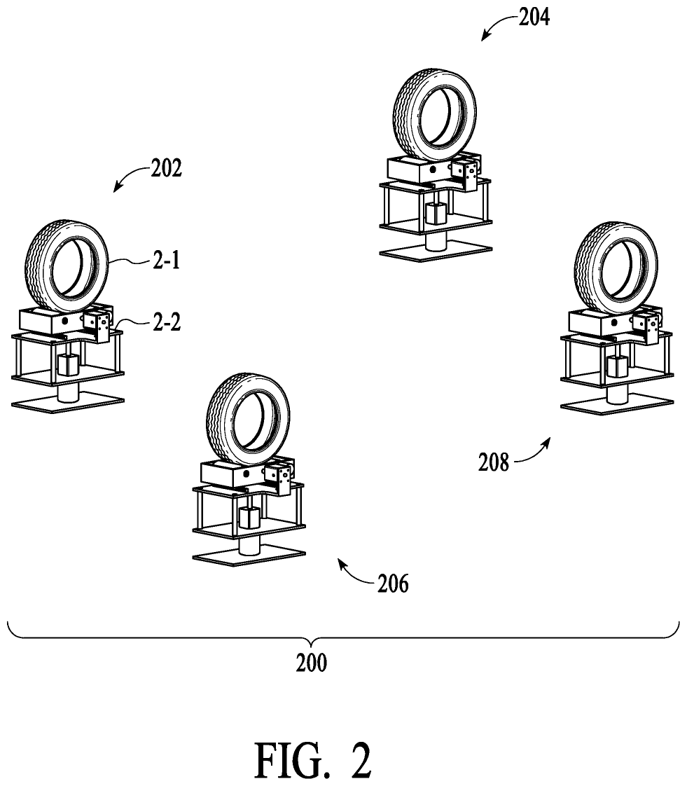

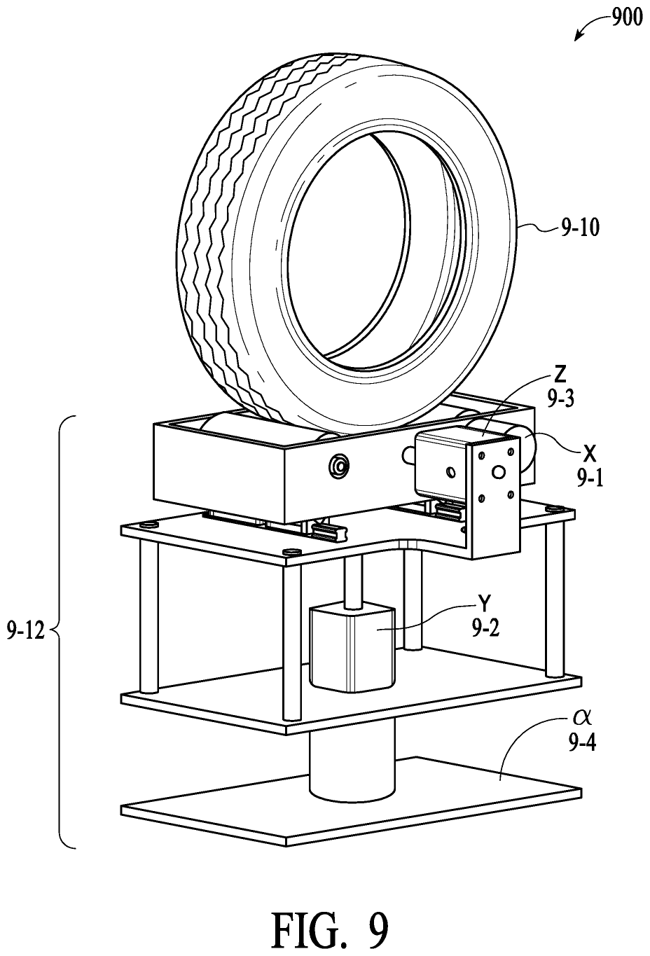

FIG. 2 shows an example of a vehicle-simulator contact setup 200. Vehicle contact means (2-1) can be a wheel, and simulator contact means (2-2) can be a special receptacle that securely holds the wheel. FIG. 2 also shows that for a vehicle with four wheels, four different simulators 202, 204, 206 and 208. Further detail of a simulator (also called vehicle AST—Autonomous Simulator and Tester) are described with reference to FIG. 9. AST replaces actual environment of operation for an AOS 100 and provides verification & validation services.

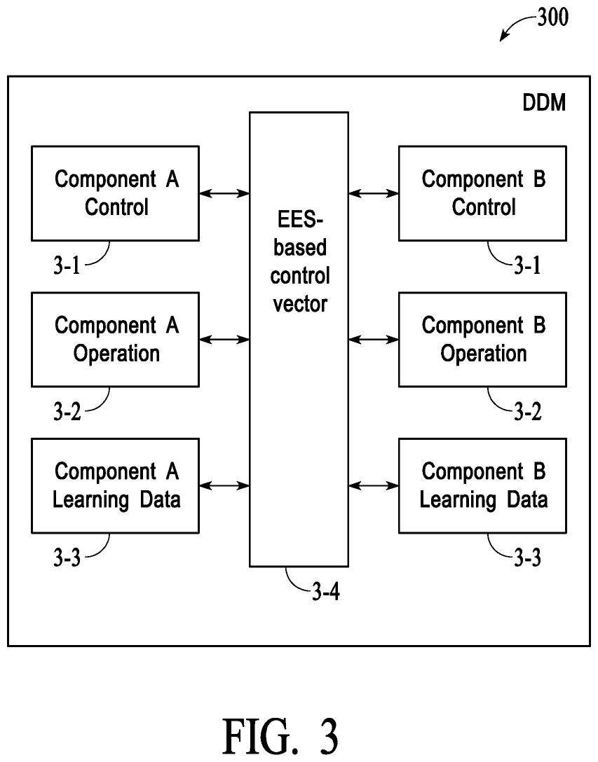

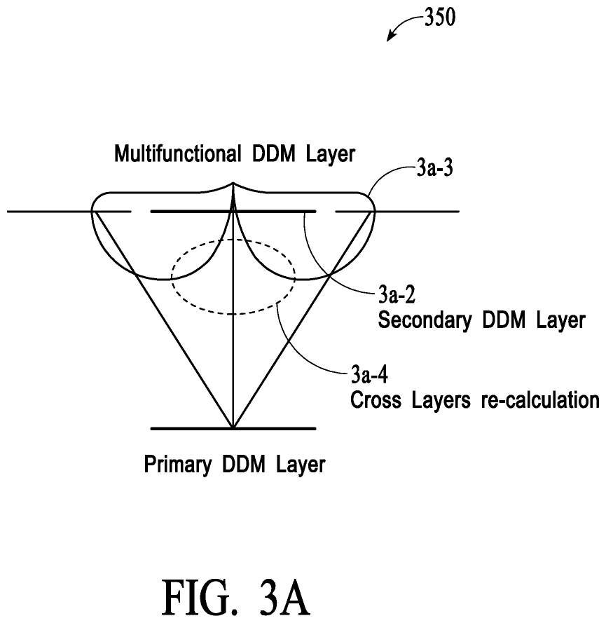

FIG. 3 shows the building blocks of a “Double Dynamic Model” (DDM) 300. Two physical components, namely Component A and Component B, are represented by two dynamic multi-parametric models. Components A and B interact at one or more active points (“work points”) at an interface that is called an Energy Exchange Platform that has a processor that runs the Energy Exchange System (EES) algorithm. In an example, Component A can be Actuation & Power-train (A&P) of an AOS 100 and Component B can be Actuation & Power-train of Autonomous Simulators & Testers (AST). Each of the components A and B comprises three simultaneous functions: EES based Control (3-1), Operation (3-2) and EES based Learning (3-3). Two-component interactive functionality is controlled by EES based Control Vector (3-4) that the processor at the Energy Exchange Platform generates. DDM then re-calculates parameters of the models of the Component A and Component B to be able to predict and regulate dynamic behavior of the AOS 100. The re-calculation is based on Control Vector (3-4). DDM can work in two configurations: Normal operation configuration and Safety operation configuration.

FIG. 3A shows a multi-functional DDM 350 representing 2+i (i=0,1,2,..) layers for multi-functional system, where each layer is full DDM structured and subjected to inter-system re-calculation (3a-4). A primary DDM layer is coupled to the multi-functional secondary DDM layer 3a-2. In the secondary DDM layer, there are intra-layer interactions 3a-3.

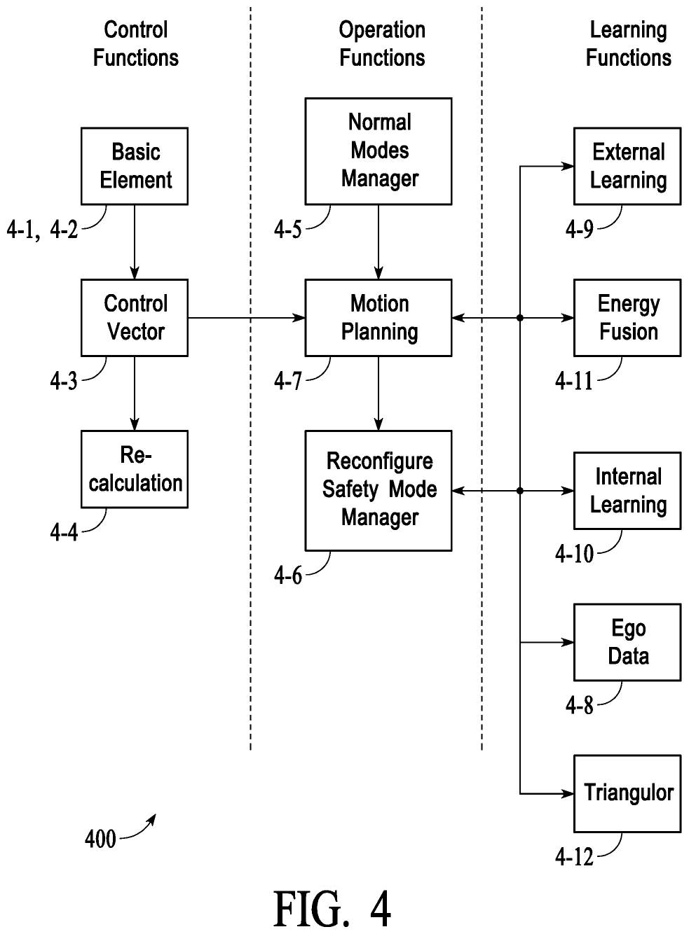

FIG. 4 shows a generic functional configuration 400 with the most significant building blocks from each of DDM components. Among the control functions, there are Clusters of “Basic Elements” (i.e. basic set of parametric elements) associated with actuators of two kinds: Sensorless Actuators (4-1) and Calculation Actuators (4-2), Control Vector (part of EES platform) (4-3), Re-calculation matrix (part of EES Platform) (4-4)—for control functionality. Among the operation functions, there is a “Motion Planning” (4-7) block, which may be integrated with navigation. Operation can be in Normal Operation Modes (continuous controllability), or Safety Operation Modes. Normal operation mode is controlled by “Normal Modes Manager” (4-5), and safety mode is controlled by a reconfigured safety mode manager (4-6). The “Learning” functions include Energy Fusion data (4-11), “Ego Data” (integrated with navigation) (4-8), internal learning data (4-10), external learning (4-9), “triangular learning” data (4-12). All these data go into motion planning (4-7) in either normal operation mode, or safety operation mode.

EES provide control platform for each one of two components structure, using “control vector”—group of mechanical parameters in physical “contact means” between two components—and, using minimal number of “sensor-less actuators” and additional “calculated actuators”, providing “continuous controllability/observability of actuation units and system. See patents U.S. Pat. No. 8,332,071B2, U.S. Pat. No. 8,958,919B2, U.S. Pat. No. 9,897,985B2 for details on sensorless actuators and EES. Each sensorless actuator output contains two measured parameters—velocity and force—represent active energy related values.

Force and velocity are vectors, and the product of the values of vectors components in parallel directions defined: 1) active power value and 2) basic parameters for computation of all related parameters and time function of them. Note that single power value is a product of single velocity value and summary of forces values, relevant to calculated direction. EES may be implemented by clusters of any actuators size and quantity, having number of active points (“work points”) of complete “actuation sub-system”. Complete “actuation sub-system” contain control (platform) means, includes “control vector” (4-3) of clusters output parameters and re-calculation matrix (4-4).

Operation functionality of DDM for each one of controlled components (note: environment component is non-controlled) based on specific configuration for each one of application, and provides “operation modes” of two kinds—“Normal Operation Modes” and “Safety Operation modes”. “Normal operation modes” executed by continuously performance of “control steps”, using “multi-parameter control vector”, “control step” re-calculation and all actuators synchronous activations. “Safety operation modes” executed, mainly, for avoiding of problem events of “safety critical” and/or “safety catastrophic” categories and followed re-configuration into pre-defined “recovery modes”, moved up to “Normal operation mode” of reconfigured component. Note that in normal operation regimes all controlled parameters values are within normal ranges and provides continuous operation. In safety operation regimes one (or more) of the controlled parameters values are out of normal operation range (pre-safety or safety zone), characterized by discontinuity, and, after subjection to recovery operation, go to normalization.

Operation functionality of DDM contain “Co-operated modes” of “two-components” performance, using, specific for each application, algorithm based mechanism, that includes, but not limited to: 1) behavior priority—example: component A-master/component B-slave, 2) priority period—examples: constant control loop time, changing of each one of control loop times, 3) condition limitation, etc.

Safety operation modes (4-6) of DDM: using ESS based control, learning and re-configuration modes, the portion of problem events—for “safety related parametric values”—subjected to “stochastic re-calculation and analysis” for “safety critical” or “safety catastrophic” categories recognition and corrective action activation. Corrective actions performed—for each one of “safety critical” or “safety catastrophic” problem events—by re-configuration for avoiding criticality.

“Motion planning” (4-7) provide managing functionality of each one of the components for operation modes exchange, using pre-defined DDM.

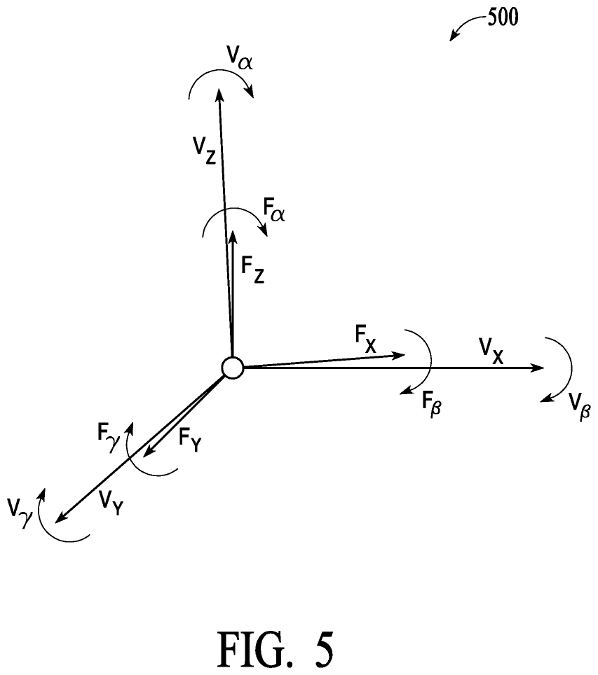

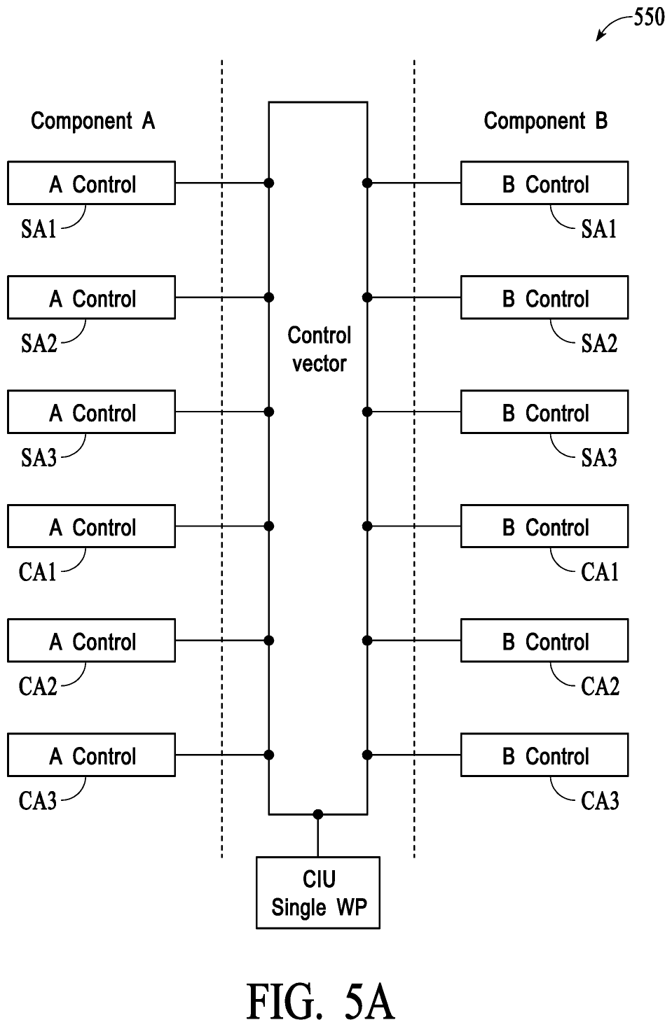

The structures of autonomous motion system (AMS) applications comprises clusters of actuators which provides basic set of parametric elements for control vector calculation by DDM. The basic set of parametric elements (see FIG. 5) are measured at “active points” (“work points”), where an active point on physical contact surface represents a point that provides “concentrated” values of the basic set of parametric elements instead of distributed values over the contact surface. One or more sensorless actuators (SA) are coupled at each of the one or more active points to enable mechanical energy exchange between the first component and the second component. Each sensor-less actuator output contains two measured parameters—velocity and force—which represent active energy related values, i.e. values indicative of mechanical energy exchange between Component A and Component B interfacing at the Energy Exchange Platform shown in FIG. 3.

FIG. 5 shows control data received from each of the sensorless actuators (SA), including basic set of parametric elements for force and velocity in six active directions (also called “energy exchange directions”) expressed as a set of parameters in the coordinate system 500, including parameters in three linear directions and three angular directions. One or more calculated actuator (CA) models are associated with each of the one or more active points. Each of the CAs acts in cooperation with the SA. The basic set of parametric elements along the three linear directions include: force along x axis (Fx), velocity along x axis (Vx), force along y axis (Fy), velocity along y axis (Vy), force along z axis (Fz), and velocity along z axis (Vz). The basic set of parametric elements along the three angular directions include: force along α angle (Fα), velocity along α angle (Vα), force along β angle (Fβ), velocity along β angle (Vβ), force along γ angle (Fγ), and velocity along γ angle (Vγ). So in summary, two “actuators clusters” (SA and CA) provide the set of twelve basic parametric elements, each one with six active directions shown in FIG. 5.

FIG. 5A illustrates a Central Integrated Unit (CIU) 550 for a single active point (also referred to as “work point”), according to an embodiment of the present disclosure. The CIU can be part of the Energy Exchange Platform at the interface of Component A and Component B. The control vector calculated by the CIU is based on data received from sensorless actuators SA1, SA2 and SA3 from both Component A and Component B, as well as data received from calculated actuators CA1, CA2 and CA3 from both components A and B.

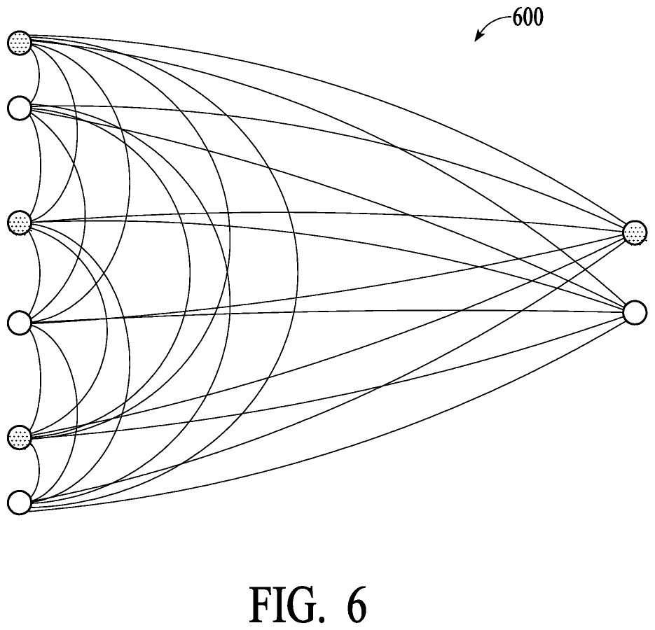

Referring back to FIG. 4, learning function can utilize basic learning model 600 shown in FIG. 6, where the EES platform uses the “Control Vector” parameters and performs continuous “full dataset” re-calculations—internal and external for each component. Additionally, each component application can utilizes special learning models for different purposes—for example: “Triangular Learning” (4-12).

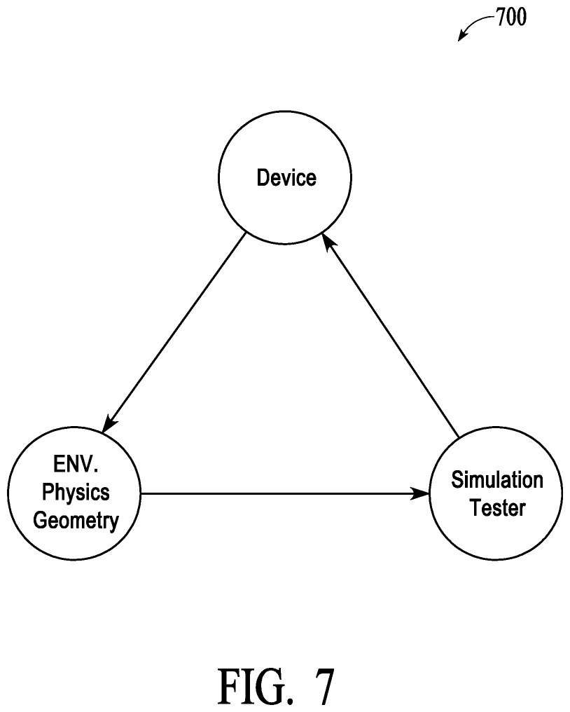

Triangular Learning model 700, shown in FIG. 7, adopts “three step cycling”. DDM operation system collects information on “Actual Environment” (e.g., physics, geometry) at active points on each control step. This data can be utilized also to create new capabilities, including external objects capabilities updating and upgrading, like: maps of cross landing, Autonomous Simulators/Testers etc.

### Vehicle A&P

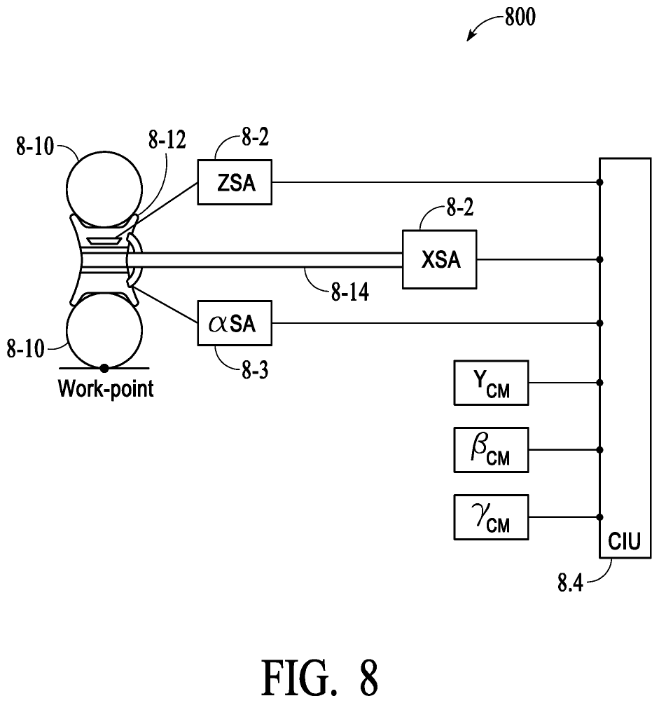

FIG. 8 shows Vehicle Actuators & Power-train (Vehicle A&P) sub-system 800 for autonomous electrical vehicles, using DDM, where Vehicle A&P can be thought of Component A. Vehicles A&P may have electrical components—e.g., motors, power electronics and computer/software means (except power supply), that are connected to input command means and power supply means, using wiring and/or internal/external communication components.

Vehicles A&P perform “energy exchange” at active points to provide motion in accordance with navigation sub-system command inputs for autonomous and/or human driving or remote-controlled driving.

Vehicles A&P performs synchronous motion of all vehicle wheels—may be from 2-to-N. In this description N=4 is assumed for a four-wheeled vehicle, as shown in FIG. 2. FIG. 8 shows a longitudinal cross section of one of the wheel tires (8-10), interfacing road, off-road or simulated road tester at the work point.

Each one of the wheels tires and rims is a part of “actuation sub-system” with general configuration of “Basic Element” of 6 active directions. Each model of Vehicle A&P has different A&P configuration. In the example in FIG. 8, most of “actuation sub-system” elements are EES adapted, using the wheel frame 812 and axle 8.14. Each wheel “basic element” comprises: X axis sensor-less actuator (XSA: 8-1), Z axis sensor-less actuator (ZSA:8-2), α-angle sensor-less actuator (αSA: 8-3), Y axis calculation module (YCM), β-angle calculation module (βCM), γ-angle calculation module (γCM), going to the Central Intelligent Unit (8-4). Note that though FIG. 8 shows input coming to CIU only from one wheel, CIU receives data from all wheels, and includes respective DDM modules for each wheel.

The CIU performs functionality of the system shown in FIG. 4. DDM structure comprises: 1) EES adapted Control function with Basic Elements, Control Vector and Re-Calculation, 2) Operation function with Operation Modes, Motion co-Planning (with Navigation sub-system), Re-Configuration (safety), 3) Learning function with Energy Fusion, External Learning, Internal Learning, Ego Data.

Vehicles A&P provides: a) continuous “deterministic” parametrical re-calculation, using full parametrical “control vector” for Normal DDM, and b) detection and classification of random problem events, “stochastic” analytics and “safety critical” events identification and followed avoiding, mainly by re-configuration using Safety DDM.

Car wheels “contact” with road and/or off-road (“road” and “off-road” includes any possible situation for any kind of cars) by interface means of wheels in road and/or off-road “contact points”, centralized in “work points”, where provide {X-Y-Z, α, β, γ & v, F} parametric control vectors—set of 12 parameters for each “work point”—Vx, Fx, Vy, Fy, Vz, Fz, Vα, Fα, Vβ, Fβ, Vγ, Fγ. of (FIG. 5). (X-Y-Z-α-β-γ) are the good known car motion directions and (v, F) are energy related parameters in each motion direction. Vehicles A&P contact means are regular tires.

Vehicles A&P comprises number of wheels, for example 4 wheels. Each wheel in work point provides three active actuation directions— X, Y, and a, that provides six actuators measured parameters—Vx, Fx, Vy, Fy, Vα, Fα of control vector. Additional parametric control vector six parameters: Vz, Fz, Vβ, Fβ, Vγ, Fγ, are calculated and executed, using three active actuators combined performance for z, β and γ control, and bring wheel control vector to “full parametric set” with “continuous control” of 12 wheel parameters for each wheel, and (12 times 4 equal to) 48 Vehicles A&P parameters for 4 wheels. If number of wheels is N, the number of parameters in 12N in this example.

XSA (8-1) contain controlled (by driver or machine controller with velocity sensor) electrical machine, coupled to “transmission”—from the machine axle 8.14 to work point—and after integration of “actuator control module”, collected data from, machine axle placed, velocity sensor and provide EES related “sensor-less actuator” after adaptation of EES calibration procedures.

ZSA (8-2) is a “vertical” oriented sensor-less actuator mainly for vibration compensation, working in parallel with other features.

αSA (8-3) is an angle actuator for synchronous direction changes of 2 or 4 wheels. αSA contain data about angle limited transmission from machine axle to work point.

YCM, βCM and γCM are Y, β, γ axis with two parameters—velocity and force-calculation modules, that manage said parameters changing by activation of XSA, ZSA and αSA.

Central Intelligent Unit (8.4) of Vehicles A&P is equivalent to the system 400 in FIG. 4, the integrated unit of all software modules of Vehicles A&P with Generic Functional Configuration items. The control functions are:

Control Vector (EES item)—12 parameters for each “work point” provides: system outputs, DDM interface, control loop and safety zone parameters.
Basic Elements (DDM items)—12 axis power actuation value.
Re-calculation matrix (EES and DDM item)—data base around “Control Vector”
Operation modes can be continuous for Normal DDM, discrete for Safety DDM. Operation functions include:
Motion Planning—allocation of Navigation and Actuation commands.
Safety Re-configuration—back-up items for self-safe solutions.
Energy Fusion—usage of energy related data for special purposes. Vehicles A&P “work points”/continuously controlled summary forces.
Learning functions include:
Internal learning—Vehicles A&P energy related parameters correlations.
External learning—road and/or off-road energy related—physical and geometry—parametrical correlations/impacts.
Ego Data—fast energy related parameters, safety zone positions, quality of performance

Note that the vehicle A&P provides general “system-in-loop” and specific “vehicle-in-loop” solutions.

### Vehicle AST

Vehicle Autonomous Simulator/Tester (AST) system 900 is for Vehicles testing in road and/or off-road simulated conditions that are dynamic, unknown a priori. AST can be the Component B in a DDM system shown in FIG. 3. In testing with road and/or off-road simulated environment, Vehicles AST and Vehicles A&P (which can be component A) co-operate continuously, using DDM.

Vehicles AST system 900 makes contact with the wheels at a number of simulated “wheel—road” contact points (see FIG. 2) by Simulator/Testing Unit (STU).

FIG. 9 shows an STU 9-12 in general configuration contains four Sensor-less Actuators (SA) at each active point in X, Y, Z and a directions and two calculation modules (CM) for regulation at active points in B and y directions. STU provides “full parametric set” of 12 parameters for each active point”: {X, Y, Z, a, B, y & V, F} control vector, V and F being Velocity and Force respectively

STU contains X axis actuator (XSA) (9-1), Y axis actuator (YSA) (9-2), Z axis actuator (ZSA) (9-3), α-angle actuator (αSA) (9-4) generating data to send to Central Intelligent Unit of component B). STU contact means can be two cylindrical rolls (which can function as master roll and co-bounded slave roll) both placed into a rigid “bathtub”-like mechanical structure.

X-SA (9-1)—controlled (by driver and machine controller with velocity sensor) electrical machine, coupled to “transmission” (from machine axle to the active point—machine rotated mechanical components) and “X-SA Controller”.

Y-SA (9-2)—controlled (by driver and machine controller with velocity sensor) electrical machine, coupled to “transmission” (bathtub body from bottom plate and rolls—up to active point) and incorporated in STU.

Z-SA (9-3)—controlled (by driver and machine controller with velocity sensor) electrical machine, coupled to “transmission” (bathtub body from side plate and rolls—up to active points) and incorporated in STU.

α-SA (9-4)—controlled (by driver and machine controller with velocity sensor) electrical machine, coupled to “transmission” (from bottom plate—up to “contact points”) and incorporated in STU.

βCM: β—incline around x in x-y plot. {ΔVB, ΔFB} control used for operation purposes up to “{Vβ, Fβ} “safe limited position” and followed “safe critical zone”. For both periods provides re-calculation of parameters in each one of the contact points.

γCM: γ—incline around z in y-z plot. {ΔVγ, ΔFγ} control used for operation purposes up to “{Vγ, Fγ} “safe limited position” and followed by “safe critical zone”. For both periods provides re-calculation of parameters in each one of the contact points.

Central Intelligent Unit for component B (i.e. AST) integrated unit of software modules of Vehicles AST, shown in Generic Functional Configuration of FIG. 4. The control functions are:

Control Vector (EES item)—12 parameters for each “work point”′ provides: simulations/tests system outputs, DDM interface, Vehicles AST control loop and safety zone parameters.
Basic Elements (DDM items)—2×6 axis power actuation values.
Re-calculation matrix (EES item)—data around “Control Vector”

Operation modes include autonomous procedures of UUT (Unit-Under-Test), simulating/testing programs and procedures—continuous for Normal DDM, discrete for Safety DDM. Operation functions include:

Motion Planning—road/off-road scenarios, testing programs and procedures.
Safety Re-configuration—back-up items for self-safe solutions.

Learning functions include:

Energy Fusion—actuators commands calculation algorithm. Simulation and testing forces pairs may be safety limited.
Internal learning—Vehicles AST energy related parameters correlations.
External learning—Vehicles A&P energy related parametrical correlations, testing and learning impacts.
Ego Data—test results.

Operation of the Vehicle AST are as follows.

UUT receiving—communication, data exchange, physical contact, EES loaded calibration, simulation/test program.

Testing execution—testing procedures, simulation scenarios, problems management—re-configuration:

a) Normal Operation: re-configuration regimes inherently includes in control loop,
b) Safety critical or catastrophic situations pre-determined for re-configuration mode for avoiding each one of at least 48 safety critical values of control vectors parameters correction activated until the criticality will rejected.

As the UUT, autonomous/automatic vehicle actuation sub-system represented by:

a) Input parametric vector {±Δv, ±Δα} received from one of next: 1) human driving means, 2) AI driving means, 3) human&AI driving means.

UUT inputs limitation for testing may be determined by:

### 0<V<Vmax, ΔVmin<ΔV<ΔVmax.

### −Amin<α<+Amax, 0<Δα<ΔAmax.

Output parametric “control vector” basic element set contains:

{ΔVx1, ΔFx1, ΔVy1, ΔFy1, ΔVz1, ΔFz1, ΔVα1, ΔFα1, ΔVβ1, ΔFβ1, ΔVγ1, ΔFγ1} (wheel 1 contact point), {ΔVx2, . . . , ΔFγ2} (wheel 2 contact point), . . . , (wheel 3 contact point), . . . (wheel 4 contact point)}.

Parametric ranges contain three zones: normal zone, pre-safe critical zone and safe-critical zone. Normal zone: normal values of all control vector parameters in all normal operation modes regimes—Pᵢ, (i=1, . . . , 48 for 4 wheels). Safe-critical zone: safety-critical values of all control vector parameters. Pre-safe critical zone: values of all control vector parameters for all safety re-configuration modes.

Vehicles AST can simulate any road/off-road impacts and other external mechanical impacts. Special “worst case” simulation regimes (scenarios) for each one of “Safety tested” parameter. Main simulation items: a) mathematical interpretation for each one of the parameters, b) SA, STU and Vehicles AST algorithms calculations.

Vehicles AST perform large scale of testing. The major testing categories: functional, endurance, safety, reliability. The tests conducted simultaneously with UUT fast specified or random navigation/driving commands—followed UUT actuation performance—and Vehicles AST simulation of road and/or environmental physical and geometry impacts. Vehicles AST testing plurality: any desired test specification & any desired test complexity & any desired test phase (development, qualification, evaluation, production, licensing) & any desired test category (functional, endurance, environmental, reliability, safety).

Vehicles AST provide, among others, safety testing of UUT, which is a dynamic system with α-priory unknown behavior. UUT can be: autonomous, manual or hybrid. For Verification & Validation purposes: each one of the safety critical parameters is subjected to worst case parametric test (except parameters of “catastrophic” category, that must be avoided a priori), using Vehicles AST continuous observability capability. Safety testing with simulated safety test conditions provides close to maximum platform for critical problems avoiding. Example: verification of navigation commands: each one of the two input parameters performed by closed to maximum similar values of output parameters of each one of the wheels. Criteria for verification of input parameters may be determined, using different calculation methods.

Safety estimation of UUT is performed by validation of “critical” parameters, subjected to combination of different “design” values of validated parameter (severity), different “exposure” simulated levels, and/or different “controllability” level scales.

Proposed methods based on good known definition for safety assessment, where the general form for safety estimations is: Safety=Severity×(Exposure×Controllability). Full safety estimation is possible based on DDM on-line calculation, using EES based control, Vehicles AST and Vehicles A&P learning and Vehicles AST abilities: DDM's “continuous observability” feature is associated with the “Controllability level.” DDM's “parametric ranges” are associated with “Severity level”, and the AST's “Road and/or Off-Road” condition simulation is associated with “Exposure level”.

“Problem Events” is a situation, where one or more of the controlled parameters values are out of normal operation range and/or characterizer by discontinuity. Such values of controlled parameters are subjected to classification and scaling procedures. The portion of problem events, that contain safety related values, are subjected to on-line stochastic re-calculation and analysis for “safety critical” or “safety catastrophic” categories and corrective action activation. Corrective actions are performed—for each one of “safety critical” events—by re-configuration for avoiding criticality. Examples are discussed below.

A) tire damage>three wheels low speed parking>damaged tire replacement
B) ΔVinput≠ΔV*output>different tasks for improvement purposes.

### Surgical A&P

Surgical Actuation & Power-train sub-system (Surgical A&P) for autonomous surgical systems can be Component A for a DDM System.

Surgical A&P has electrical components similar to Vehicle A&P as described above. The Surgical A&P can be part of the Autonomous Operation System (AOS) (11-1) shown in FIG. 11, which is coupled to the Surgical AST (11-2), that is described below.

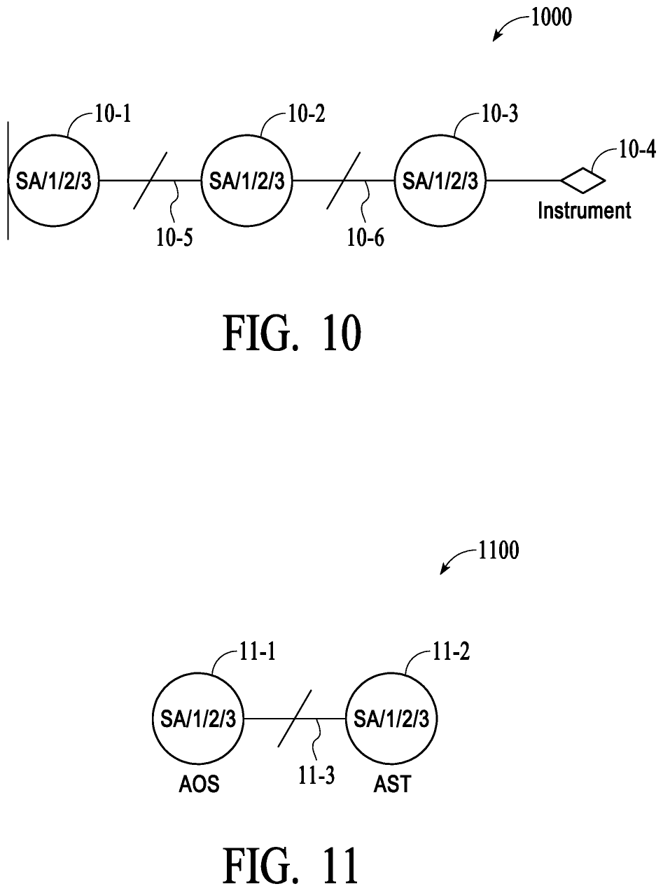

Surgical A&P perform controlled functionality for a surgical instrument (such as 10-4 shown in FIG. 10) at a plurality of “work points” including controlled instrument transportation to “work point” for the purpose of providing any desired specific motion depending on tissue structure parameters in the work environment of the surgical instrument. In other words, the surgical instrument can provide mechanical motion (11-3) at “work point” and every controlled step may have to reconfigure unknown tissue structure rather than assuming that the work environment will remain unchanged. The surgical instrument often has to be structurally flexible with motion parameters at the work points changing continually.

Note that the surgical A&P 1000 can have multiple sections, as shown in FIG. 10, each section can have its “section work points” and the instrument as a whole can have one or more work points. In the example shown in FIG. 10, surgical A&P has sections 10-5, 10-6 for mechanical energy exchange and the section closest to instrument head 10-4. Multi-functional DDM shown in FIG. 3A may be used for a multi-section configuration like this.

Each one of the sections' motion can be controlled in 6 active directions. Surgical A&P have two typical section heads: 1) Instrument Head and 2) Position Head, with basic configuration of three sensorless actuators (SA): XSA, αSA, βSA (labeled as SA1/2/3) and three calculated actuators (CA): YSA, ZSA, γSA (labeled as CA1/2/3). Each one of the “Heads” is a part of “actuation sub-system” with general configuration of “Basic Element” and are EES adapted. Computer/software Central Intelligent Unit CIU related to all lead sections and includes DDM software means. For each section, there is the three SAs, as shown at 10-1, 10-2 and 10-3.

Surgical A&P provides: a) continuous “deterministic” parametrical re-calculation, using full parametrical set via control vector—for Normal DDM, and b) detection and classification of random problem events, “stochastic” analytics and “safety critical” events identification and followed avoiding, mainly by re-configuration—for Safety DDM.

Surgical A&P contacts body tissues using different instruments in “work points”, providing {X, Y, Z, α, β, γ & V, F}parametric control vectors, which is a set of 12 parameters for each “work point”—Vx, Fx, Vy, Fy, Vz, Fz, Vα, Fα, Vβ, Fβ, Vγ, Fγ (as in FIG. 5). X-Y-Z-α-β-γ are the body and instrument motion directions and “V, F” are energy related parameters, Velocity and Force respectively, in each motion direction.

Surgical A&P used number of instruments. Each instrument in work point provides control vector of “full parametric set” with “continuous control” of 12 parameters.

Central Intelligent Unit of Surgical A&P is the integration unit for software modules of as shown in FIG. 4. Control functions include:

Control Vector (EES based)
Basic Elements (DDM i—2×6 axis power actuation values.
Re-calculation matrix (EES based, DDM executed)

Operation modes include continuous for Normal DDM, discrete for Safety DDM.

### Motion Planning.

Safety Re-configuration—back-up items for self-safe solutions.

Learning functions include:

Energy Fusion—usage of energy related data for special purposes. Surgical A&P “work points”/continuously controlled DDM summary forces.
Internal learning—Surgical A&P energy related parameters correlations.
External learning—energy related—physical and geometry—parametrical correlations/impacts.
Ego Data—fast energy related parameters, safety zone positions, quality of performance

### Surgical AST

Surgical Autonomous Simulator/Tester 1100, shown in FIG. 11, can be component B coupled with component A, which can be an autonomous operation system (AOS), such as the surgical A&P shown in FIG. 10. Surgical AST simulates conditions for the Actuation & Power-train (Surgical A&P) to be tested under dynamic condition of changing tissue environment.

Surgical AST and Surgical A&P co-operated continuously, using DDM. Note that tissue may re-structures with unknown behavior and change of geometrical and physical parameters. “Instrument Head” of Surgical A&P and is mechanically connected Surgical AST along a flexible interface

Computation functionality of Surgical AST includes: a) simulations & functional testing of “Instrument Head” with “Position Head” disconnected, b) simulations & functional testing of “Position Head” with SA's and CA's of “Instrument Head” disconnected, c) “Flexible Leads” simulations & testing plurality (functional, endurance, safety, reliability).

The simulation test unit (STU) of Surgical AST have computer/software means connected to the Central Intelligent Unit—each one of the actuators and STUs are integrated in centralized software for Surgical AST control, operation and learning using DDM and Multi-functional DDM.

Central Intelligent Unit of Surgical A&P is the integration unit for software modules of as shown in FIG. 4. Control functions include:

Control Vector (EES based)
Basic Elements (DDM based)—power actuation values.
Re-calculation matrix (EES based)—data base around “Control Vector”

Operation modes for the UUT (Unit-Under-Test) simulating/testing programs and procedures includes continuous for Normal DDM, discrete for Safety DDM.

Motion Planning—simulation scenarios, testing programs and procedures.
Safety Re-configuration—back-up items for self-safe solutions.

Learning functions include:

Energy Fusion—actuators commands and calculation algorithm.
Internal learning—Surgical AST energy related parameters correlations.
External learning—Surgical A&P energy related parametrical correlations, testing and learning impacts.
Ego Data—test results.

UUT receives communication, data exchange, physical contact, EES calibrations, simulation/test programs.

Testing execution involves

a) testing procedures, simulation scenarios, problems management—re-configuration:
b) Safety critical or catastrophic situations detection, re-configuration for problem situations avoiding,

UUT in autonomous/automatic surgical actuation sub-system represented by:

Input parametric vector {±Av, ±Aa} received from one of next: 1) human driving means, 2) AI driving means, 3) human&AI driving means.

UUT inputs limitation for testing may be determined by:

### 0<V<Vmax, ΔVmin<ΔV<ΔVmax.

### −Amin<α<+Amax, 0<Δα<ΔAmax.

Output parametric “control vector” contains: {ΔVx1, ΔFx1, ΔVy1, ΔFy1, ΔVz1, ΔFz1, ΔVα1, ΔFα1, ΔVβ1, ΔFβ1, ΔVγ1, ΔFγ1} (tissue contact point#1), {ΔVx2, . . . , ΔFγ2} (tissue contact point#2), . . . , (tissue contact point#3)}.

Parametric ranges contain three zones: normal zone, pre-safe critical zone and safe-critical zone. Normal zone: normal values of all control vector parameters in all normal operation modes regimes. Safe-critical zone: safety-critical values of all control vector parameters. Pre-safe critical zone: values of all control vector parameters for all safety re-configuration modes.

Surgical AST can simulate any organ mechanical impacts. Special “worst case” simulation regimes (scenarios) for each one of “Safety tested” parameters. Main simulation items include: a) mathematical interpretation for each one of the axes, c) SA, STU and Surgical AST algorithms calculations.

SurgicalAST performs large scale of testing. The major testing categories are: functional, endurance, safety, and reliability. The tests conducted simultaneously with UUT fast “specified or random” navigation driving commands, followed by UUT test actuation performance and Surgical AST simulation of tissue physical and/or geometry impacts.

Surgical AST can also handle any desired test specification & any desired test complexity & any desired test phase (development, qualification, evaluation, production, licensing) & any desired test category (functional, endurance, environmental, reliability, safety).

Safety estimation of UUT is performed by performing validation of “critical” parameters one by one, subjected to combination of: a) different “design” values of validated parameter (severity), b) different “exposure” simulated levels, c) different scales of “controllability” level.

Proposed methods based on good known definition for safety assessment—“Safety=Severity×(Exposure×Controllability)—the general form for safety estimations. Surgical AST can provide full safety estimation, based DDM on-line calculation, using EES based control, SurgicalAST and SurgicalA&P learning abilities:

a. “continuous observability” accounts for “Controllability level”
b. Parametric ranges account for “Severity level”
c. Tissue and Bones condition simulates for “Exposure level”

“Problem Events” situations arise when one or more of the controlled parameters values are out of normal operation range and/or characterized by discontinuity. Such values of each one of controlled parameters are subjected to classification and scaling procedures. The portion of problem events may comprise “safety related values”—subjected to on-line stochastic re-calculation and analysis for “safety critical” or “safety catastrophic” categories and corrective action activation. Corrective actions performed for each one of “safety critical” or “safety catastrophic” problem events by re-configuration for avoiding criticality.

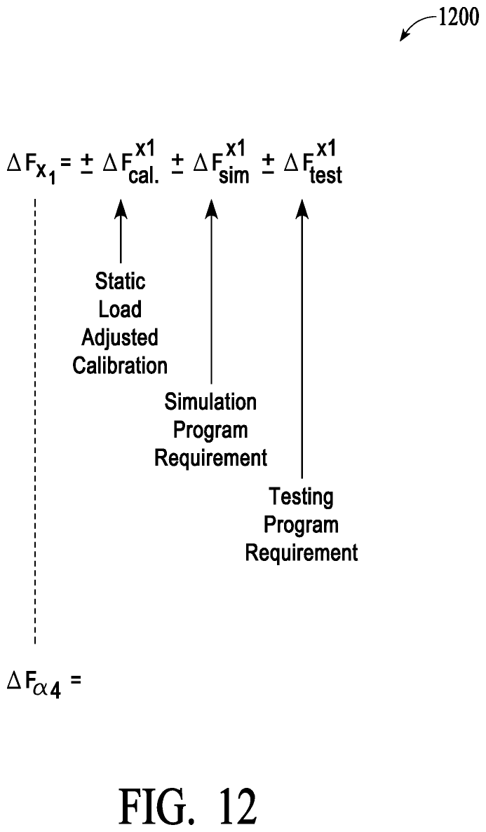

FIG. 12 shows the simulation and Test commands calculation. Only the Force data for X linear axis for just one work point is shown here, but as discussed above, force data is collected from multiple work points in multiple directions are fed into the EES system for control vector calculation. For example, in the autonomous vehicle safety testing (shown in FIG. 2), Fx1 is force data from the first wheel along X linear axis. For the first wheel, force data from Y and Z linear axes and α, β and γ angular axes are also collected. Similarly force data for X, Y, Z linear axes and α, β and γ angular axes are collected from all four wheels. Note that here number of wheels N=4, but any number of wheels can be accommodated for collecting data. Similarly, velocity data can be collected too from the multiple directions for each work point. A required value for EES to perform its calculation is calibration data adjusted for static load. Additionally, the data includes simulation program requirement that includes inputs for AST simulation program implementation. Finally, the data includes testing program requirement that includes inputs for AST testing program implementation.

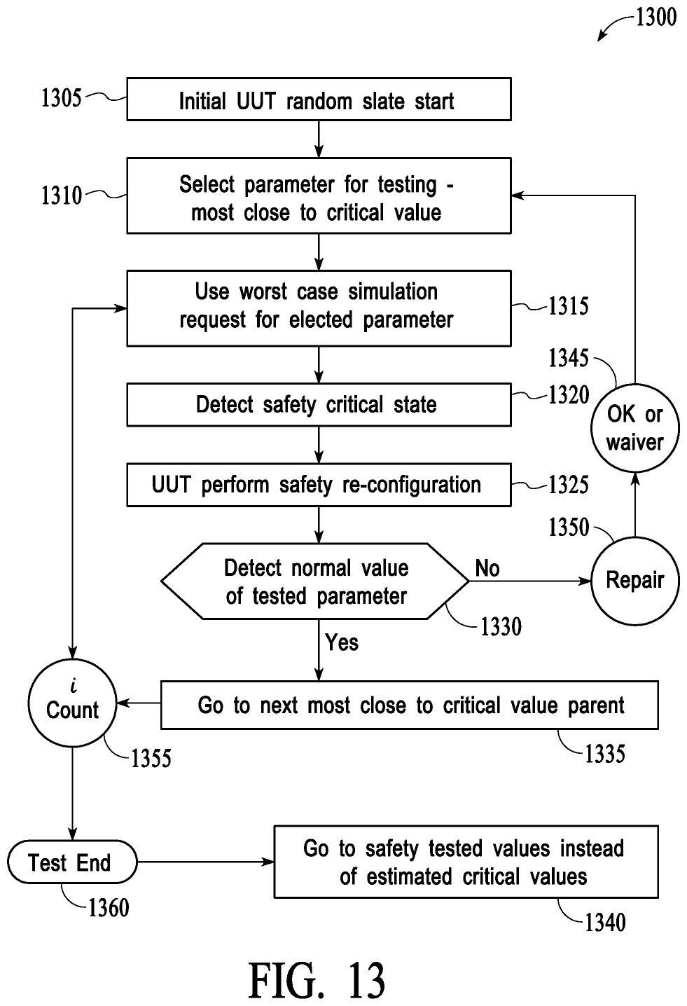

FIG. 13 illustrates a flowchart 1300 describing a method for safety testing, according to an embodiment of the present disclosure. Note that the steps of the flowchart do not have to be performed in order. More steps now shown in FIG. 13 may be added, or some steps shown in FIG. 13 may not be needed in all scenarios.

The method starts with a “random slate” starting point for initial UUT (1305). At 1310, parameters for testing are selected. The parameters whose values are most close to known critical values are chosen. At 1315, for each of the selected parameters, worst case simulation is requested. At 1320, a “safety critical” state is detected. At 1325, DDM performs safety re-configuration for the UUT. After reconfiguration, if “normal” (i.e., not safety-critical) values of selected parameters are detected at 1330, then at 1335, the next value of the parameter is chosen that is next close to the previous “critical value”. If, on the other hand, “normal” (i.e., not safety-critical) values of selected parameters are not detected at 1330, then appropriate “repair” functions are called upon at 1350. Note that the repair requirement can be waived at 1345.

From 1335, for each count at 1355, the worst case simulation request for the next parameter is requested. After all the parameters are finished, the test ends at 1360, and the system goes to safety mode from the critical mode at 1340, i.e. the system adopts safety tested values instead of estimated values.

### Computer System Environment

The disclosed methods and systems are implemented in the form of a machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system includes a processing device, a main memory (e.g., read-only memory (ROM), flash memory, dynamic random access memory (DRAM) such as synchronous DRAM (SDRAM) etc.), a static memory (e.g., flash memory, static random access memory (SRAM), etc.), and a data storage device, which communicate with each other via a bus.

Processing device represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 602 may also be one or more special-purpose processing devices such as an application specific integrated circuit (ASIC), a field programmable gate array (FPGA), a digital signal processor (DSP), network processor, or the like. The processing device is configured to execute instructions for performing the operations and steps discussed herein.

The computer system may further include a network interface device to communicate over the network. The computer system also may include a video display unit (e.g., a liquid crystal display (LCD) or a cathode ray tube (CRT)), an alphanumeric input device (e.g., a keyboard), a cursor control device (e.g., a mouse or a touch pad),), a signal generation device (e.g., a speaker), a graphics processing unit, video processing unit, and audio processing unit.

The data storage device may include a machine-readable storage medium (also known as a computer-readable medium) on which is stored one or more sets of instructions or software embodying any one or more of the methodologies or functions described herein. The instructions may also reside, completely or at least partially, within the main memory and/or within the processing device during execution thereof by the computer system, the main memory and the processing device also constituting machine-readable storage media.

In one implementation, the instructions include instructions to implement functionality corresponding to a height difference determination. While the machine-readable storage medium in an example implementation can be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “obtaining” or “associating” or “executing” or “generating” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general purpose systems may be used with specific programs in accordance with the teachings herein, or it may prove convenient to construct a more specialized apparatus to perform the method. The structure for a variety of these systems will appear as set forth in the description below. In addition, the present disclosure is not described with reference to any particular programming language. It will be appreciated that a variety of programming languages may be used to implement the teachings of the disclosure as described.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing specification, implementations of the disclosure have been described with reference to specific example implementations thereof. It will be evident that various modifications can be made thereto without departing from the broader spirit and scope of implementations of the disclosure as set forth in the following claims. The specification and drawings are, accordingly, to be regarded in an illustrative sense rather than a restrictive sense.

1. A system for autonomous motion control, comprising:
a first component and a second component that interact with each other at one or more active points at an interface of the first component and the second component, wherein each of the first component and the second component is represented by a respective dynamic multi-parametric model, wherein the first component is part of an autonomous motion system (AMS) and the second component simulates an environment in which the AMS operates in;
an energy exchange platform at the interface that receives control data, operation data and learning data from each of the first component and the second component, and generates, by a processor at the energy exchange platform, a control vector associated with mechanical energy exchange at the one or more active points due to the interaction of the first component and the second component,
wherein the processor further recalculates parameters of the respective dynamic multi-parametric models of the first component and the second component using the control vector as a baseline, thereby autonomously predicting and regulating dynamic behavior of the AMS.

2. The system of claim 1, wherein the processor of the energy exchange platform iteratively recalculates the parameters in multiple layers of a double dynamic model (DDM) that involves cross-layer re-calculation.

3. The system of claim 1, wherein one or more sensorless actuators (SA) are coupled at each of the one or more active points to enable mechanical energy exchange between the first component and the second component.

4. The system of claim 3, wherein control data received from the sensorless actuators includes a basic set of parametric elements for force and velocity in six active directions, including three linear directions and three angular directions.

5. The system of claim 4, the basic set of parametric elements along the three linear directions include: force along x axis (Fx), velocity along x axis (Vx), force along y axis (Fy), velocity along y axis (Vy), force along z axis (Fz), and velocity along z axis (Vz).

6. The system of claim 5, the basic set of parametric elements along the three angular directions include: force along α angle (Fα), velocity along α angle (Vα), force along β angle (Fβ), velocity along β angle (Vβ), force along γ angle (Fγ), and velocity along γ angle (Vγ).

7. The system of claim 4, wherein one or more calculated actuator (CA) models are associated with each of the one or more active points.

8. The system of claim 7, wherein the processor generates the control vector based on control data received from each of the sensorless actuators as well as each of the calculated actuator models.

9. The system of claim 1, wherein the operation data received by the energy exchange platform is used to determine whether to configure the parameters to operate the system in a normal operation mode or in a safety operation mode.

10. The system of claim 9, wherein when the system operates in a normal operation mode, the processor recalculates the parameters of the respective dynamic multi-parametric models of the first component and the second component in a deterministic way.

11. The system of claim 9, wherein when the system operates in a safety operation mode, the processor recalculates the parameters of the respective dynamic multi-parametric models of the first component and the second component in a stochastic way.

12. The system of claim 11, wherein parameters to be recalculated in the safety operation mode include parameters that are required to perform corrective actions to ensure safety.

13. The system of claim 11, wherein parameters to be recalculated in the safety operation mode include parameters that are required to perform corrective actions to prevent critical failure.

14. The system of claim 1, wherein the learning data associated with the energy exchange platform includes one or more of the following: external learning data, energy fusion data, internal learning data, and ego data.

15. The system of claim 14, wherein the learning data further includes triangular learning data.

16. The system of claim 1, wherein one of the first components and the second components operate as a master, while the other component operates as a slave.

17. The system of claim 1, wherein the first component comprises a vehicle actuator and power train (vehicle A&P) system of an autonomous vehicle.

18. The system of claim 17, wherein the second component comprises a vehicle autonomous simulation and test (vehicle AST) fixture on which a wheel of the autonomous vehicle is mounted, wherein the vehicle AST simulates a road or off-road condition for the autonomous vehicle.

19. The system of claim 1, wherein the first component comprises a surgical actuator and power train (surgical A&P) system of a robotic surgical device.

20. The system of claim 19, wherein the second component comprises a surgical autonomous simulation and test (surgical AST) fixture that simulates an environment of living tissue in which the robotic surgical device is to perform.
